(19) United States
(12) Patent Application Publication
(10) Pub. No.: US 2008/0188815 A1
(43) Pub. Date: Aug. 7, 2008

(54) Hypodermic Needle Safety Apparatus

(75) Inventor: Michael Anthony Leman Scott, Cape Town (ZA)

Correspondence Address: MORGAN, LEWIS & BOCKIUS LLP (SF), One Market, Spear Street Tower, Suite 2800, San Francisco, CA, 94105, US

(73) Assignee: Michael Anthony Leman Scott, Cape Town (ZA)

(21) Appl. No.: 11/814,873

(22) PCT Filed: Aug. 8, 2006
(86) PCT No.: PCT/IB06/02167

§ 371 Date: Jul. 26, 2007

(30) Foreign Application Priority Data
Dec. 19, 2005 (ZA) 2005/10279

(51) Int. Cl. A61M 5/32

(52) U.S. Cl. 604/198

(57) ABSTRACT

The invention concerns a hypodermic needle safety apparatus (10) which is either manufactured as a separate unit for retrofitting to an existing hypodermic needle (28), for example the hypodermic needle of a syringe, or which is connected integrally to the needle. The safety apparatus has an end cap (16) which locates over the tip of the needle when the apparatus is in a safe condition and which can be retracted along the needle during use. A flexible, resilient support structure (20) extends alongside the needle and supports the end cap. In a relaxed state the support structure has an inwardly bowed shape that prevents retraction of the end cap. A deflecting arrangement (22) is provided which can operate to deflect the support structure from the inwardly bowed shape to a shape that allows retraction of the end cap along the needle.

### BACKGROUND TO THE INVENTION

This invention relates to a hypodermic needle safety apparatus.

Accidental hypodermic needlestick injuries are a source of major concern for medical practitioners in view of possible transmission of potentially fatal infections such as HIV and Hepatitis B or C.

Numerous solutions have been proposed for reducing the risk of an accidental needlestick injury. One class of solutions requires manual intervention to cover the needle tip, for instance by recapping it after use or by withdrawing it into the barrel of a syringe. In another class of solutions, the needle tip is covered automatically on withdrawal thereof from the skin of the patient. An example of the latter class, described in U.S. Pat. No. 5,250,031, involves the use of a sheathing apparatus having a series of flexible elements extending alongside the length of the needle, from the base thereof, and supporting an end cap. When a patient is to be injected, the needle passes through an opening in the end cap. As the needle enters the patient, the flexible elements bow outwardly and retract along the length of the needle. When the needle is withdrawn from the patient, the resilience of the elements causes them to flex back towards their original shapes, thereby relocating the end cap over the tip of the needle. Additional security can then be provided by manual intervention, the arrangement being such that the end cap can be displaced sideways in order to misalign the opening and needle. This prevents the needle from being pushed out of the end cap again.

The present invention seeks to provide an alternative arrangement in which the needle tip is automatically covered on withdrawal.

### SUMMARY OF THE INVENTION

According to one aspect of the present invention there is provided a hypodermic needle safety apparatus comprising an end cap which is locatable over a tip of a hypodermic needle when the apparatus is in a safe condition and which is retractable along the needle during use of the needle, a flexibly resilient support structure for extending alongside the needle and supporting the end cap, the support structure when in a relaxed state having an inwardly bowed shape that prevents retraction of the end cap, and deflecting means operable to deflect the support structure from the inwardly bowed shape to a shape that allows retraction of the end cap along the needle.

In the preferred embodiments, the deflecting means is arranged to allow the support structure to revert to the inwardly bowed shape when the end cap has retracted a certain distance along the needle.

Typically the end cap has a passage therein in which the needle tip locates when the apparatus is in the safe condition and through which the needle extends when the end cap retracts along the needle. For added security against exposure of the needle tip when the apparatus is in the safe condition, the end cap may be movable laterally relative to the needle tip thereby to move the passage out of alignment with the needle.

Conveniently the support structure comprises a plurality of flexible elements which, in the relaxed state of the structure, are inwardly bowed towards one another. Typically there will be a pair of flexible elements located in use on opposite sides of the needle. In the case of an apparatus which can be retrofitted to an existing hypodermic needle, the apparatus may include a base hub locatable over an end of the needle remote from the needle tip, with the flexible elements extending from the base hub to the end cap. In alternative arrangements, the flexible elements may extend from a base of the needle or, where the needle is fitted to a syringe, from the syringe itself.

The deflecting means may include a resilient structure arranged to engage the flexible elements of the support structure and to flex them to an outwardly bowed shape. Such resilient structure may include a plurality of resiliently flexible arms which extend outwardly relative to the flexible elements when the arms are in a relaxed state, the arms having ends which are engagable with the elements when the arms are flexed resiliently to a loaded position in which they are nested alongside the elements, such that the arms in the loaded position apply a force to the elements which tends to bow them outwardly. In the preferred embodiment, there is a pair of arms extending from the end cap, but in other embodiments it would be possible for the arms to extend from the base hub, from a syringe to which the needle is fitted, or from the support structure itself. The arms may have ends which are shaped to engage the elements from the inside and to remain engaged with the elements while a certain amount of outward bowing of the elements takes place, and thereafter to release the elements.

The apparatus may also include a safety cap shaped to fit over the arms, elements and needle, at least when the arms are in the loaded position nested alongside the elements, thereby to maintain such nested position.

### BRIEF DESCRIPTION OF THE DRAWINGS

The invention will now be described in more detail, by way of example only, with reference to the accompanying drawings in which:

FIG. 1 shows a perspective view of an apparatus according to the invention in a relaxed state prior to loading;

FIG. 2 shows another perspective view of the apparatus in the relaxed state;

FIG. 3 shows a face view of the apparatus in the relaxed state;

FIG. 4 shows a side view of the apparatus in the relaxed state;

FIG. 5 diagrammatically illustrates the apparatus when loaded and capped by a separate safety cap; and

FIGS. 6(a) to 6(c) diagrammatically illustrate the apparatus at sequential stages of operation.

### DESCRIPTION OF THE ILLUSTRATED EMBODIMENT

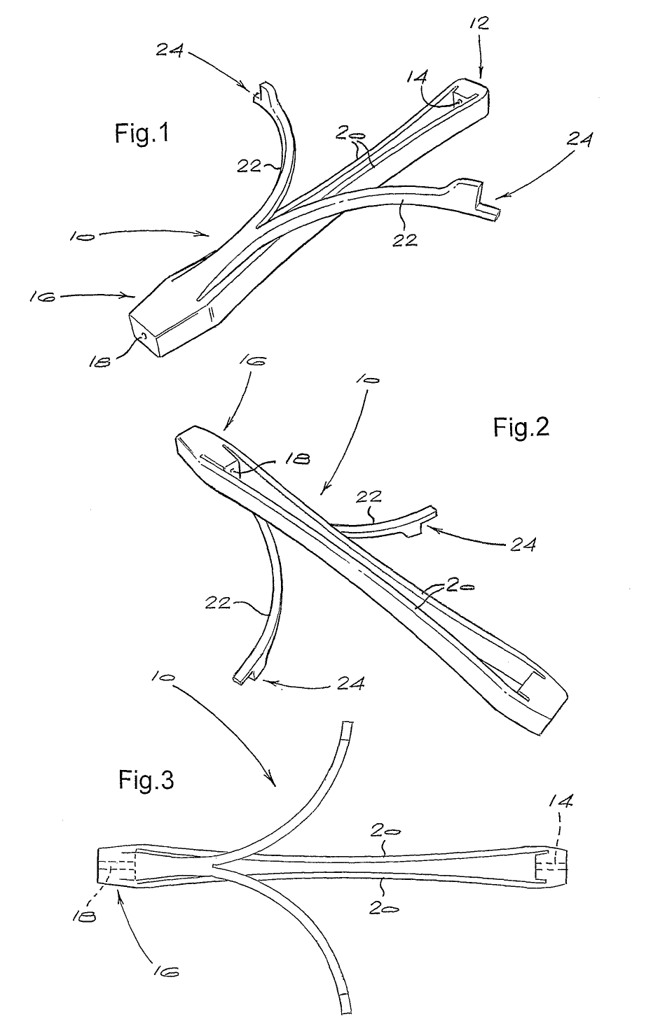

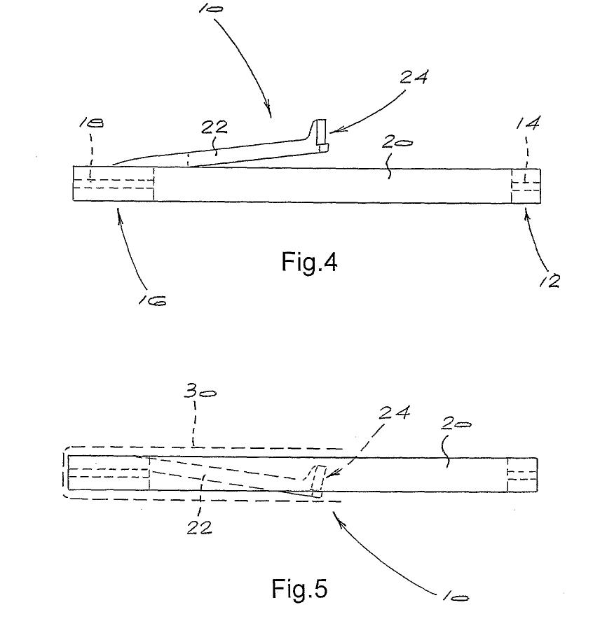

The hypodermic needle safety apparatus 10 seen in FIGS. 1 to 4 is in the form of a one-piece plastics moulding. It consists of a base hub 12 having a central passage 14, an end cap 16 having a central passage 18, a pair of elongate, slender elements 20 extending from the base hub to the end cap, and a pair of arms 22 having notched ends 24. The plastics material of which the apparatus is moulded is of such a nature that the elements 20 and arms 22 are flexibly resilient in nature.

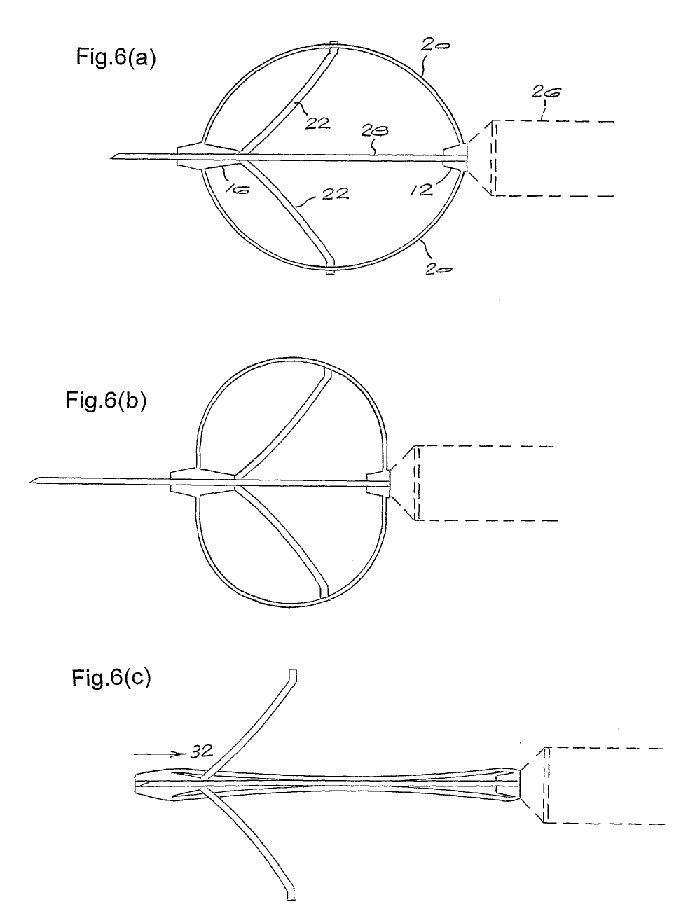

In FIG. 6(a) the numeral 26 indicates the front end of a conventional syringe fitted with a hypodermic needle 28. The apparatus 10 is retrofitted to the needle such that the needle passes through the passage 14 in the base hub 12.

FIGS. 1 to 4 illustrate the apparatus 10 in a relaxed state. In this state, the elements 20 have an inwardly bowed shape, and the dimensions of the apparatus 10 are such that the tip of the needle is located inside the passage 18 of the end cap 16.

In order to use it, the apparatus is “loaded”. This is achieved by flexing the arms 22 inwardly between the elements 20, against their natural resilience, and arranging them such that their notched ends 24 engage the elements from the inside, as shown in FIG. 5. Given that the arms project outwardly when relaxed, it will be understood that when so flexed, they apply an outward force to the elements. In this “loaded” state, with the elements 20, arms 22 and needle 28 in a nested, compact configuration alongside one another, the tip of the needle resides in the passage 18 of the end cap 16.

The apparatus is maintained in this compact, “loaded” state by placing a safety cap 30 over the end cap 16, elements 20, arms 22 and needle 28, as shown in FIG. 5. The safety cap restrains the arms 22 from springing back towards their relaxed state and maintains the apparatus in the safe condition. When the syringe is to be used, for example to draw a medicine from a vial, the cap 30 is pulled off the nested components. The arms spring outwardly towards their relaxed shapes, causing the elements 20 to assume the outwardly bowed shape seen in FIG. 6(a).

This is accompanied by some retraction of the end cap 16 over the needle, i.e. movement of the end cap towards the base hub 12, with the result that the needle projects some distance through the passage 18.

The tip of the needle may now be inserted into the vial and the syringe operated in the normal way to withdraw the medicine. The needle can then be withdrawn from the vial for subsequent insertion into a patient who is to be injected with the medicine.

Conventionally the needle is inserted a fairly substantial distance into the patient. As the needle enters the patient, the patient's skin pushes on the end cap 16 and causes further retraction thereof, accompanied by further outward bowing of the elements 20, as shown in FIG. 6(b).

The design of the arms 22, notched ends 24 and elements 20, and in particular the shape of the notched ends, is such that when the needle has been inserted a certain distance, the notched ends will automatically slip off the elements 20, thereby disengaging them. Although they are now released the elements are caused to remain in the outwardly bowed shape by the action of the patient's skin on the end cap 16 as long as the needle remains inserted. However, when the needle is withdrawn from the patient, the natural resilience of the elements 20 causes them to revert to the relaxed shape seen in FIGS. 1 to 4 and FIG. 6(c).

When the elements 20 reach their initial, relaxed shape the needle tip once again locates in the passage 18 in the end cap 16, i.e. the needle tip is automatically covered once it has been fully withdrawn from the patient.

An important feature of the apparatus is the fact that the elements 20 have an inwardly bowed shape, in which they bow inwardly towards one another, when relaxed. The relatively inflexible needle resists inward bowing of the elements beyond the relaxed state. This geometry makes it impossible to cause the end cap 16 to retract along the needle by pushing it in the direction of the arrow 32 in FIG. 6(c). In other words the shape and inherent resilience of the elements 20 enable them to lock the end cap over the needle tip, thereby reducing the chances of an accidental needlestick injury.

Syringe-mounted hypodermic needles are also frequently used to withdraw blood or other fluid samples from patients. In this case as well, commencing with the loaded condition, insertion of the needle into the patient will eventually cause the arms 22 to slip off and release the elements 20, whereafter the sequence of events described above will take place. When drawing a liquid sample from a vial or other container, it is preferable not to insert the needle so far that the stopper of the container, through which the needle is inserted, pushes the end cap 16 back far enough for the arms 22 to release the elements 20.

Were this to happen, the needle tip would be covered by the end cap when it is withdrawn from the stopper and it would be necessary to “reload” the apparatus, i.e. to re-engage the notched ends 24 of the arms with the elements 20 in order to cause them to bow them outwardly, before injecting the patient. Alternatively, inadvertent disengagement could be avoided by manually restraining the arms 22 to prevent them from releasing the elements 20 when the needle is inserted into the vial.

In the example described above, the apparatus 10 is a separate unit which can be retrofitted to an existing hypodermic needle of conventional type. It is however within the scope of the invention for the apparatus to be incorporated as an integral part of a hypodermic needle or as an integral part of a device, such as a syringe, to which the needle is mounted. It would for example be possible for the base hub to be connected to the needle or syringe body or to be integral with the syringe body. It would also be possible to do away with the base hub altogether and for the elements 20 to project integrally from the body of the syringe or from the base of the hypodermic needle itself.

In order to improve the security of the apparatus it would be possible to modify the illustrated apparatus to allow for the end cap 16 to be moved laterally relative to the needle tip after use of the syringe, such that the passage 18 is no longer aligned with the needle. The end cap 16 may for instance include a second, blind passage alongside the passage 18 into which the needle tip may be moved by laterally displacing the end cap relative to the needle.

1. A hypodermic needle safety apparatus comprising an end cap which is locatable over a tip of a hypodermic needle when the apparatus is in a safe condition and which is retractable along the needle during use of the needle, a flexibly resilient support structure for extending alongside the needle and supporting the end cap, the support structure when in a relaxed state having an inwardly bowed shape that prevents retraction of the end cap, and deflecting means operable to deflect the support structure from the inwardly bowed shape to a shape that allows retraction of the end cap along the needle.

2. An apparatus according to claim 1 wherein the deflecting means is arranged to allow the support structure to revert to the inwardly bowed shape when the end cap has retracted a certain distance along the needle.

3. An apparatus according to claim 1 wherein the end cap has a passage therein in which the needle tip locates when the apparatus is in the safe condition and through which the needle extends when the end cap retracts along the needle.

4. An apparatus according to claim 1 wherein the support structure comprises a plurality of flexible elements which, in the relaxed state of the structure, are inwardly bowed towards one another.

5. An apparatus according to claim 4 wherein the support structure comprises a pair of flexible elements located in use on opposite sides of the needle.

6. An apparatus according to claim 4 wherein the apparatus includes a base hub locatable over an end of the needle remote from the needle tip and wherein the flexible elements extend from the base hub to the end cap.

7. An apparatus according to claim 4 wherein the flexible elements extend from a base of the needle.

8. An apparatus according to claim 4 wherein the flexible elements extend from a syringe to which the hypodermic needle is mounted.

9. An apparatus according to claim 4 wherein the deflecting means includes a resilient structure arranged to engage the flexible elements of the support structure and to flex them to an outwardly bowed shape.

10. An apparatus according to claim 9 wherein the resilient structure includes a plurality of resiliently flexible arms which extend outwardly relative to the flexible elements when the arms are in a relaxed state, the arms having ends which are engageable with the elements when the arms are flexed resiliently to a loaded position in which they are nested alongside the elements, such that the arms in the loaded position apply to a force to the elements which tends to bow them outwardly.

11. An apparatus according to claim 10 comprising a pair of arms extending from the end cap.

12. An apparatus according to claim 10 wherein the arms have ends which are shaped to engage the elements from the inside and to remain engaged with the elements while a certain amount of outward bowing of the elements takes place, and thereafter to release the elements.

13. An apparatus according to claim 10 wherein the ends of the arms are notched.

14. An apparatus according to claim 10 and including a safety cap shaped to fit over the arms, elements and needle, at least when the arms are in the loaded position nested alongside the elements, thereby to maintain such nested position.

15. A hypodermic needle having mounted thereon an apparatus according to claim 1.

16. A syringe having a hypodermic needle according to claim 15.
